(19) United States
(12) Patent Application Publication
(10) Pub. No.: US 2014/0107631 A1
(43) Pub. Date: Apr. 17, 2014

(54) ANNULAR KERATOPIGMENTATION SYSTEMS AND METHODS OF VISION CORRECTION OF PRESBYOPIC EYES

(71) Applicant: Francis FERRARI, Strasbourg (FR)

(72) Inventor: Francis FERRARI, Strasbourg (FR)

(21) Appl. No.: 14/051,622

(22) Filed: Oct. 11, 2013

Related U.S. Application Data
(60) Provisional application No. 61/713,013, filed on Oct. 12, 2012.

(51) Int. Cl. A61F 9/008; A61F 9/013

(52) U.S. Cl. CPC A61F 9/00804; A61F 9/013
USPC 606/5; 606/4; 606/166

(57) ABSTRACT

Systems, devices, and methods for correcting presbyopic vision create a dye ring in the cornea. The intrastromal ring is created using a femtosecond laser and is centered on the visual axis. A black or a colored pigment is then injected. The internal diameter of the ring is dimensioned so as to create an intracorneal pinhole and improve the near and intermediate vision of the non-dominant eye while only slightly altering the distance vision of that eye. The pinhole dye ring in the cornea of the presbyopic eyes enhances the depth of field, thereby allowing improved presbyopic performance without the need for corrective lenses.

### CROSS-REFERENCE TO RELATED APPLICATION

This application claims benefit of priority of U.S. Provisional Patent Application Ser. No. 61/713,013 filed on Oct. 12, 2012, the entire disclosure of which is incorporated herein by reference.

### TECHNICAL FIELD

This technology relates to systems, devices, and methods of treating presbyopia. More particularly, the technology relates to systems, devices, and methods of creating a pigmented intrastromal ring in the cornea to enhance the depth of field, thereby providing improved presbyopic performance without the need for corrective lenses.

### BACKGROUND

Inlays have been implanted in human eyes to attempt to reverse the effects of presbyopia and to restore near and intermediate vision. For example, one available inlay (Kamra inlay) is an opaque circular micro-disc with a small opening in the center. Additionally, there are high precision, laser-etched micro-openings through the depth of the inlay to help maintain a healthy cornea.

When placed in the cornea, the small opening in the center of the inlay blocks unfocused light and only allows focused light to reach the retina. With focused light rays, patients often enjoy a wider range of improved vision. Other corneal inlays are also utilized. Corneal inlays are often small lenses inserted into the cornea to reshape the front surface of the eye to improve vision. Many of these corneal inlays are used to improve near vision and to reduce the need for reading glasses in older adults who have presbyopia.

For example, one corneal inlay (Vue+ or PresbyLens) is a 2 mm diameter inlay made of hydrogel plastic, which is similar to the material used in soft contact lenses. The inlay improves both near and intermediate vision. The inlay is placed within the cornea under a LASIK-style flap (laser assisted in situ keratomileusis). When in position, the inlay changes the curvature of the cornea so the front of the eye acts like a multifocal contact lens. Patients who had the inlay implanted in the cornea of their non-dominant eye can realize an improvement of five lines of near visual acuity and an improvement of one to two lines of intermediate visual acuity on a standard eye chart, while maintaining binocular distance vision of 20/20.

Another corneal inlay (Flexivue Microlens) uses a laser to create a tiny “pocket” just below the surface of the eye. Eye surgeons insert a microlens for correction of presbyopia. The pocket seals itself to hold the lens in place. The lens is made of hydrophilic polymer, a highly wettable synthetic substance often used in intraocular lenses that permanently replace the eye's natural lens in cataract surgery. The microlens is permanent but can be removed and replaced if a stronger prescription is needed. The microlens lens can be 3 mm in diameter and 20 microns thick at the edges.

Many of the inlays have complications as a result of the biomaterial of the inlay itself, due to the pinhole effect, and due to the incision. For example, intracorneal iron deposits form, inflammation of the interface occurs, and thinning of the stroma can result from the inlay. Further, an increase in the apoptosis caused by the inlay along with the presence of inflammatory markers in the cornea 24 to 48 hours after the surgery can also occur. Likewise, pinhole effects can include the loss of visual acuity lines for distance vision, hyperopic shift, night haloes, and monocular diplopia. Problems resulting from the incision can include ocular dryness and loss of visual acuity lines.

Additional surgical techniques have also been used to correct presbyopia, including laser blended vision, conductive keratoplasty, and refractive lens exchange. For example, laser blended vision addresses presbyopia by creating short-sightedness (myopia) in one eye (the non-dominant eye) and normal-sightedness (emmetropia) in the other eye (the dominant eye). Blended vision can be implemented by LASIK or by other laser procedures that re-shape the corneas to have one eye focused for distance and the other eye focused for near vision. The eyes will work together to produce blended vision.

Conductive keratoplasty uses radio waves to adjust the contour of the cornea by shrinking the corneal collagen around it. Conductive keratoplasty can be used to treat hyperopia, astigmatism, and presbyopia. Conductive keratoplasty is a non-invasive alternative to other types of eye surgery and uses heat energy from low-level radio frequency waves instead of a laser to shrink the corneal collagen fibers in order to steepen the cornea. After anesthetic drops have been applied and have taken affect, a probe with a special tip that transfers radio frequency energy is used to administer uniform treatment spots around the periphery area of the cornea. The heat generated by the radio frequency waves is designed to shrink the collagen of the area and to cause the cornea to steepen to a very high degree. Less regression is expected due to the uniform delivery of heat and deep shrinkage of collagen.

Refractive lens exchange is a surgical procedure that involves removing the natural lens in the eye and replacing it with a tiny permanent prescription intraocular lens resulting in improved vision and reduced dependency on glasses or contact lenses. This technique is different than many other refractive surgeries, like LASIK, that involve reshaping the cornea. A single-vision intraocular lens can be inserted to eliminate nearsightedness, farsightedness, and astigmatism. In this scenario, a patient's distance vision will be significantly improved, but the patient will still need reading glasses. Recently, multifocal intraocular lenses have been developed to correct distance and reading vision.

Other techniques for treating presbyopia include multi-focal approaches, including contact lenses with two (or more) distinct lens powers. For example, some multi-focal contact lenses have a bifocal design with two lens powers—one for distance vision and one for near vision. Others have a multifocal design similar to progressive eyeglass lenses, with a gradual change in lens power for a progressive visual transition from distance to close up. The multifocal designs can include a concentric bifocal pattern with the near correction in a small circle at the center of the lens, surrounded by a larger circle containing the distance correction.

Alternating image designs (also called translating designs) have distinct zones in the lens for distance vision and near vision. These designs are typically available in gas permeable (GP) lens materials only. Like bifocal glasses, the top part of an alternating image multifocal GP lens is for distance vision and the bottom part is for near vision. The two zones are separated by a nearly invisible line that helps an eye care professional determine if the lens is fitting properly. When a patient looks straight ahead while wearing an alternating multifocal lens, the patient is looking through the distance portion of the GP lens. When the patient looks down to read, the lens remains supported by the patient's lower lid, so the patient's line of sight now passes through the lower (near vision) portion of the lens.

The near segment can have a half-moon, crescent or annular shape. (The annular segment circles around the entire periphery of the lens.) In alternating multifocals with half-moon or crescent-shaped near segments, the lens maintains its proper rotational position by means of an area of unequal thickness in the lens called a prism ballast. In some cases, the bottom edge of the lens is also truncated to help align it properly with the wearer's lower lid.

Because alternating multifocal lenses typically have just two lens powers, these lenses usually provide good vision for driving and for reading. However, they may not perform as well as simultaneous image designs for computer work and other intermediate-range visual tasks.

For example, simultaneous image designs have both distance and near vision portions of the lens in front of the pupil at the same time. These designs are available in both soft lens and GP lens materials. The wearer's brain must determine which area of the lens to emphasize and which area to ignore to provide the best image resolution.

In addition to these methods of attempting to treat presbyopia, corneal tattooing has been used separately to conceal corneal scarring or to conceal a white cataract by applying dyes on a cornea that has been cauterized. Other non-perforating micro-needle treatments have been used for the cosmetic treatment of leucoma. Similarly, others have treated the opaque cornea of patients by introducing a dye in a corneal pocket that had been previously pre-dissected. Keratopigmentation has been used to treat iris defects with the use of new pigments and purified and inert dyes that no longer interact with neighboring tissues.

### SUMMARY

None of the previous techniques and systems provides a permanent, safe, and affordable solution to presbyopia. Multifocal lenses can shift in the wearer's eye, while implants often fail to provide improved distance vision, especially in low light conditions. Implants often result in difficulty in focusing, dry eyes, and haloing at night. Implants are foreign bodies, and it is unknown or can be difficult to predict how the cornea will react to the implants. For example, vision may appear hazy or unclear, and the implants may cause inflammation of the eye.

The systems and methods of the claimed annular keratopigmentation invention create an intrastromal ring centered on the visual axis into which a colored pigment is injected. The internal diameter of the intrastromal ring is dimensioned to create a pinhole and to improve the vision of the eye.

The systems and methods of the claimed invention create a pinhole ring (black or colored) in the cornea of presbyopic eyes to enhance the depth of field, thereby providing improved presbyopic performance without the need for corrective lenses, implants, or a change in the corneal curvature with a laser procedure and avoiding many of the associated problems. In the methods of the claimed invention, an intrastromal ring is created with a femtosecond laser, and at least one side port is created with the laser in the periphery of the ring (or radially to the ring). Dye is injected in the cornea via the side port(s) to create a pigmented ring in the stroma. The pigmented ring treats presbyopia by blocking unfocused light and allowing focused light to reach the retina. Image sharpness increases as the effective size of the pupil decreases.

The systems, devices, and methods of the claimed invention use a keratopigmentation technique to create a black (or colored), concentric, intrastromal ring centered on the visual axis, with an internal diameter of less than 2 mm, so as to create a pinhole to improve presbyopic performance.

### BRIEF DESCRIPTION OF THE DRAWINGS

FIGS. 1A and 1B illustrate an intrastromal ring created in a cornea with side ports and radial ports, respectively.

FIGS. 1C-1K show the creation of an intrastromal ring in the cornea using the ports and a PresbyRing spatula in accordance with the claimed invention.

FIG. 2A shows an example front view of a clockwise spatula in accordance with the claimed invention.

FIG. 2B shows an example side view of a clockwise spatula and a counterclockwise spatula in accordance with the claimed invention.

FIG. 2C shows an example front view of a counterclockwise spatula in accordance with the claimed invention.

FIG. 3 shows dye injected through a port into an intrastromal ring in accordance with the claimed invention.

FIGS. 4A-4C illustrate another example embodiment of the claimed invention where the PresbyRing method with regard to FIGS. 1-3 is combined with a LASIK procedure to provide additional vision correction.

FIG. 5 shows dye injected through a port into an intrastromal ring in the example combined embodiment of the claimed invention.

FIG. 6 shows a flap returned to its original position in the example combined embodiment of the claimed invention.

FIG. 7 illustrates another example embodiment of the claimed invention where a FemtoRing method is used to change a patient's eye color.

FIGS. 8A-8C illustrate an example method to remove a pigmented intrastromal (PresbyRing) from a cornea using an excimer laser.

FIG. 9 shows an example eye treated with a femtosecond laser to create tunnels for intracorneal rings in accordance with the claimed invention.

FIG. 10 shows dye injected in the two tunnels created in FIG. 9 in accordance with an example embodiment of the claimed invention.

FIG. 11 shows the result of an example method of the claimed invention where a ring is created using the femtosecond laser, followed by dye completely injected in the two tunnels.

FIG. 12A shows an eye treated at 250 μm without injection of dye.

FIG. 12B shows diffused light and retro illumination of the eye shown in FIG. 12A.

FIG. 12C shows Optical Coherence Tomography Spectral Domain (OCT-SD) spectrolysis results of the eye of FIGS. 12A-12B.

FIGS. 13A-13B shows a result of a method of the claimed invention where a continuous pigmented layer is located along the incision of an eye, which does not diffuse in the adjacent stroma.

FIG. 13C illustrates a linear hyper-reflective line located along the incision interface and associated with a posterior shadow in an eye treated with a method of the claimed invention.

FIG. 13D shows a spectral domain OCT of the depth of incision of an eye treated with a method of the claimed invention.

FIGS. 14A-14D show examples of an intrastromal ring centered on the visual axis using a method of keratopigmentation in accordance with the claimed invention.

### DETAILED DESCRIPTION

The systems and methods of the claimed invention create a pinhole ring (black or colored) in the cornea of presbyopic eyes to enhance the patient's depth of field, thereby providing improved presbyopic performance without the need for corrective lenses, implants, or a change in the corneal curvature with a laser procedure. The methods of the claimed invention eliminate insertion of an intracorneal ring-shaped inlay by using keratopigmentation directly. In the methods of the claimed invention, an intrastromal ring and one or two side ports are created with a femtosecond laser. Additional side ports can also be created, as needed. The side ports are created in the periphery of the ring (or radially to the ring). Dye is injected in the cornea via the side ports to create a pigmented ring in the stroma. The pigmented ring treats presbyopia by blocking unfocused light and allowing focused light to reach the retina. Image sharpness increases as the effective size of the pupil decreases.

A number of methods using a variety of the devices and systems in accordance with the claimed invention can be performed depending upon the patient and the diagnosis. A PresbyRing method can be performed with or without a combined LASIK (laser-assisted in situ keratomileusis) or PRK (photorefractive keratectomy) treatment. Additionally, a FemtoRing method can also be performed to change the color of a patient's eyes using a similar technique.

### PresbyRing Method

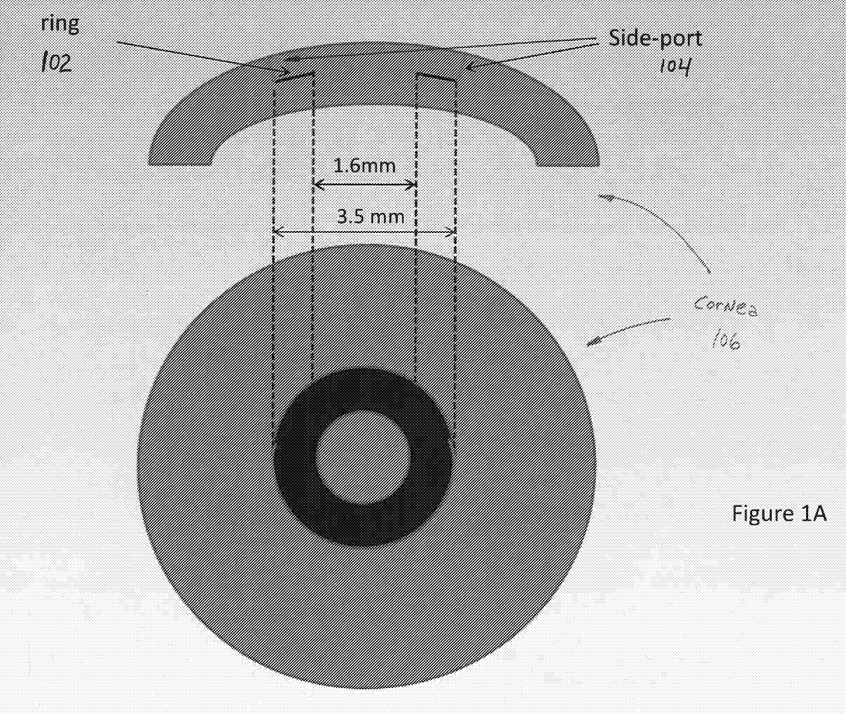

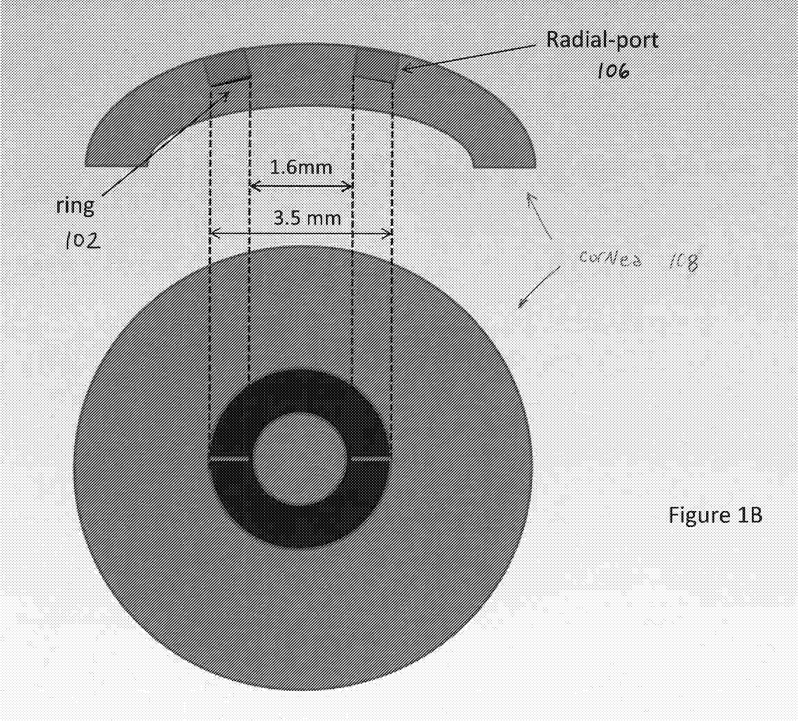

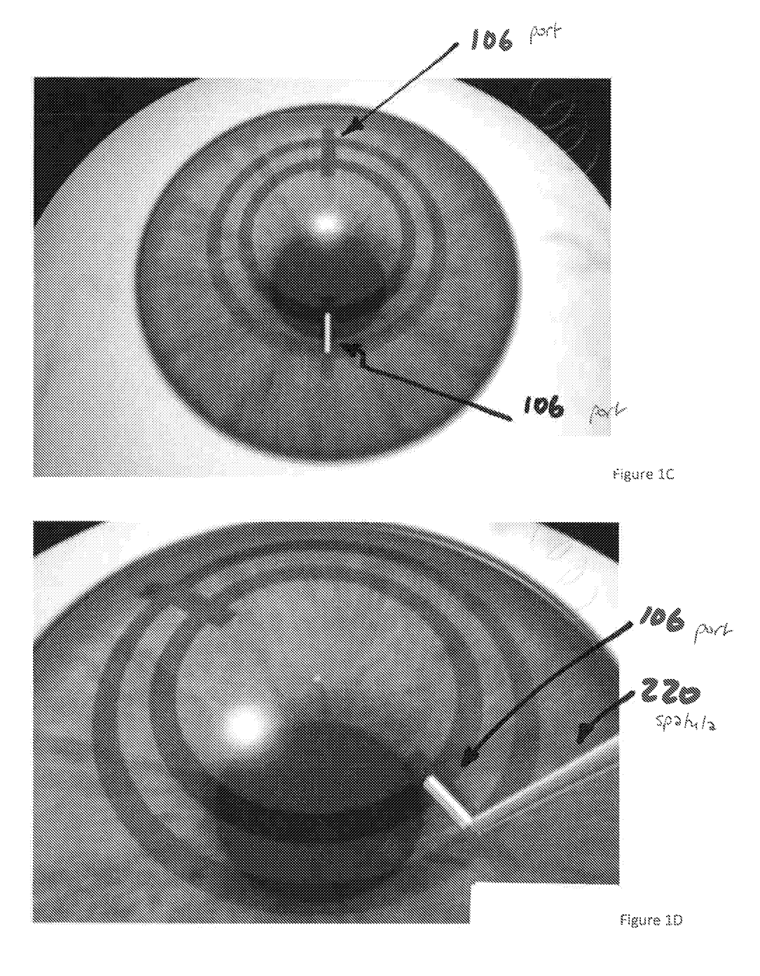

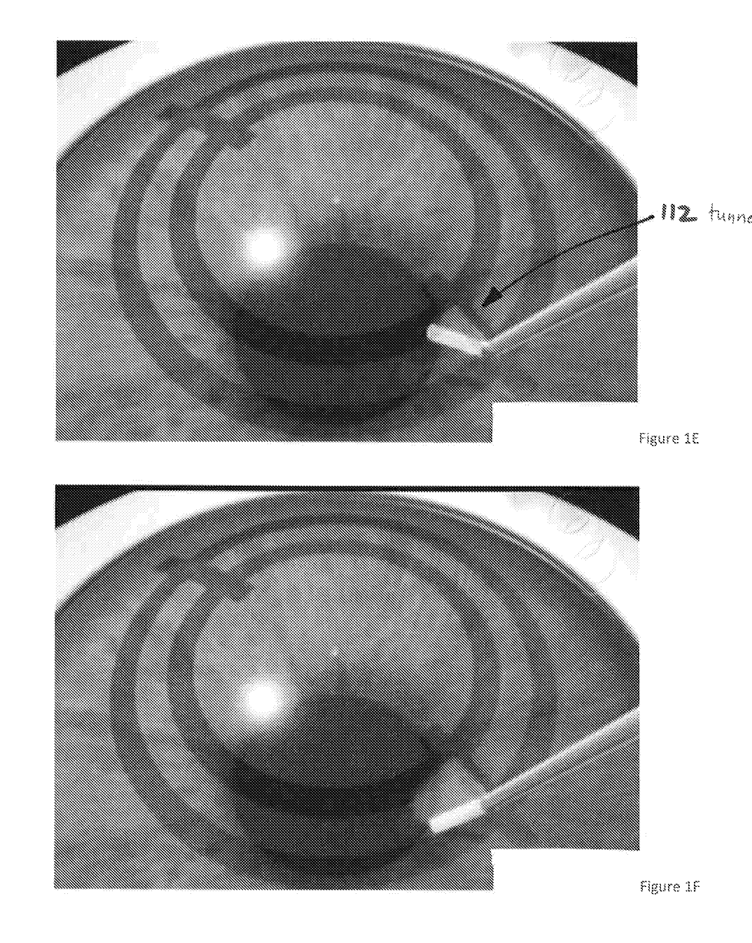

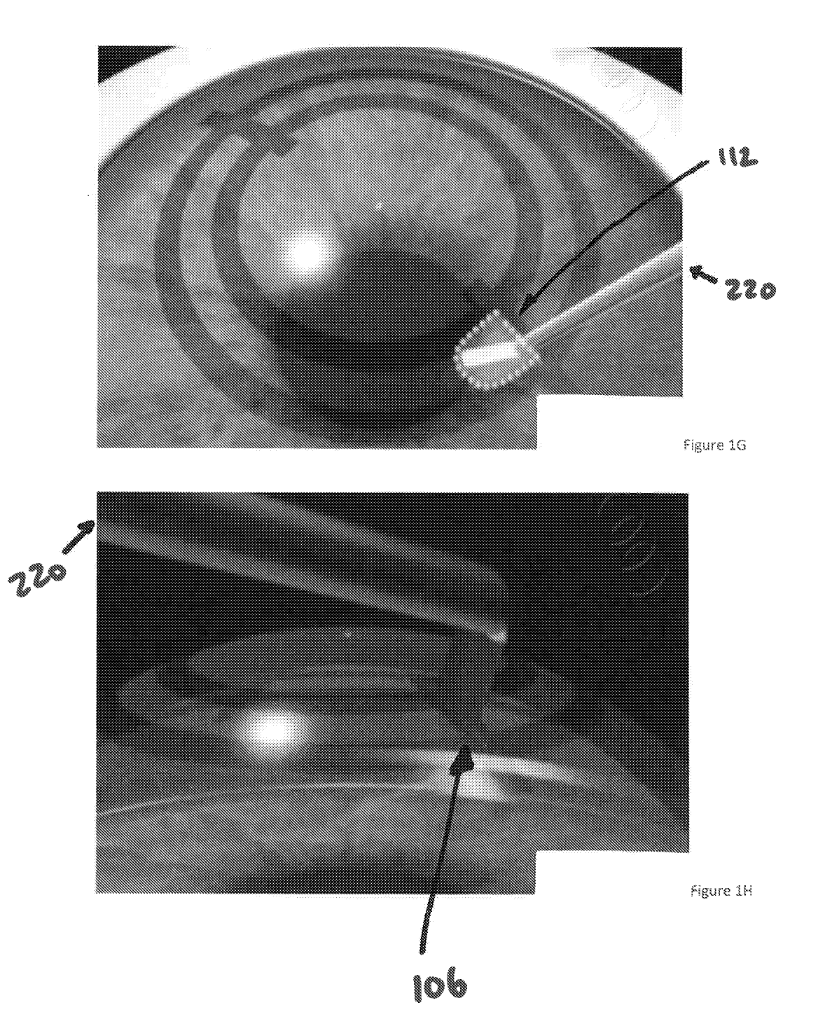

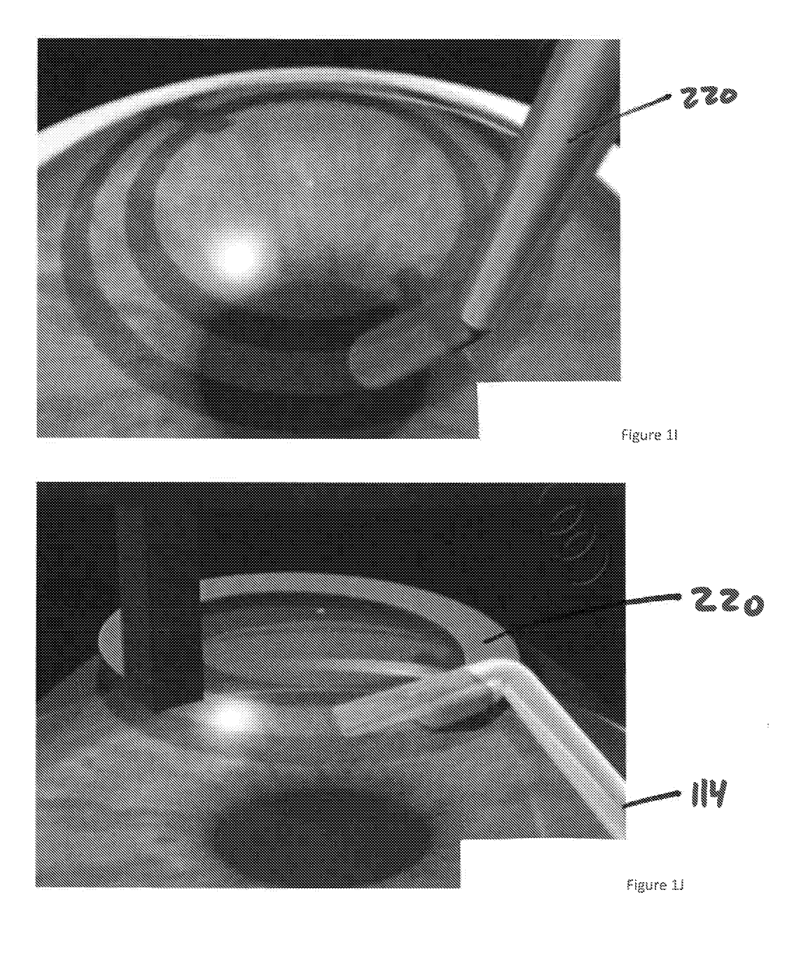

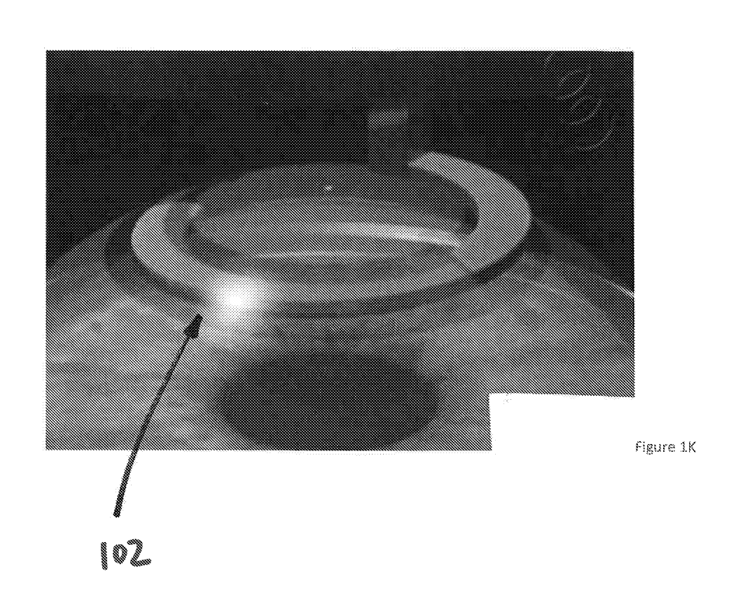

One method in accordance with the claimed invention includes positioning a patient on a laser bed. Topical anesthesia is given to the patient. The bed is moved under a femtosecond laser. A lid speculum is inserted, and the eye's cornea is brought in contact with the femtosecond laser's cone. As shown in FIGS. 1A and 1B, an intrastromal ring 102 is created with a femtosecond laser, and one or two side ports 104 are created in the cornea 108 with the laser (not shown separately). The side ports 104 are created by laser incision substantially concentric with the visual axis and at right angles (vertically) to the cornea 108. The radial ports 106 are created by laser incision substantially radial to the visual axis and at right angles (vertically) to the cornea 108. The ring 102 and the side ports 104 (or radial ports 106) can be made with a femtosecond laser, such as a Carl Zeiss Meditec Visumax® laser.

The depth of the ring 102 can be determined by measuring the thickness of the cornea. The depth of the ring 102 can be between 5 microns and 500 microns, for example. In one example embodiment, the ring 102 is between 100 microns and 400 microns. In another example embodiment, the ring 102 is placed at a depth of 75% of the thickness of the cornea 108. In one example embodiment, the ring 102 has a 1.6 mm internal diameter and a 3.8 mm external diameter. In the example embodiments shown in FIGS. 1A and 1B, the external diameter of the ring 102 is 3.5 mm. The dimensions and the depth of the ring 102 can be modified depending upon the patient and the characteristics of the patient's eye(s). FIG. 1A illustrates ring 102 with dye injected and side ports 104, and FIG. 1B shows ring 102 with dye injected and radial ports 106.

As shown in FIG. 1C (and further in FIGS. 1D-1K), after one or two side ports 104 (or radial ports 106) are created with the laser, the patient can be removed from the cone and the eye can be examined under a microscope. A PresbyRing spatula 220, as shown in FIG. 2A, is inserted through the ports 104, 106 in FIG. 1D to create at least one tunnel 112 as shown in FIGS. 1E-1G. The tunnel(s) 112 is expanded and used to create the ring 102. For example, spatula 220 is inserted vertically in the port 104, 106 as shown in FIG. 1H. The angle of the spatula 220 is changed to a more horizontal position as shown in FIG. 1I. In the horizontal position, the spatula 220 can be used to enlarge the tunnel(s) 112 into a ring 102. Additionally, as shown in FIGS. 1J and 1K, a guide 114 can be used to assist in keeping the port 104, 106 open as a spatula 220 is inserted into the tunnel 112 to create a ring 102.

FIGS. 2A and 2C show example front views of a clockwise spatula 220 and a counter-clockwise spatula 230, respectively, with an internal diameter of 1.8 mm and an external diameter of 3.6 mm. FIG. 2B shows a side view 226 of the two spatulas. As outlined above with regard to FIGS. 1D-1K, the two ring surfaces of the cornea 108 created with the laser are separated using the two PresbyRing spatulas 220, 230 to create a ring-shaped volume. In order to create the tunnel(s) 112, one example embodiment of the claimed invention uses successively a clockwise spatula and a counter-clockwise spatula. Other geometries and dimensions of the spatulas can be used depending upon the shape and dimension of the tunnel to be created. For example, FIG. 2B shows a side image of a right angle spatula 226 with a handle 227 at 90 degrees to the separating portion 228 of the spatula.

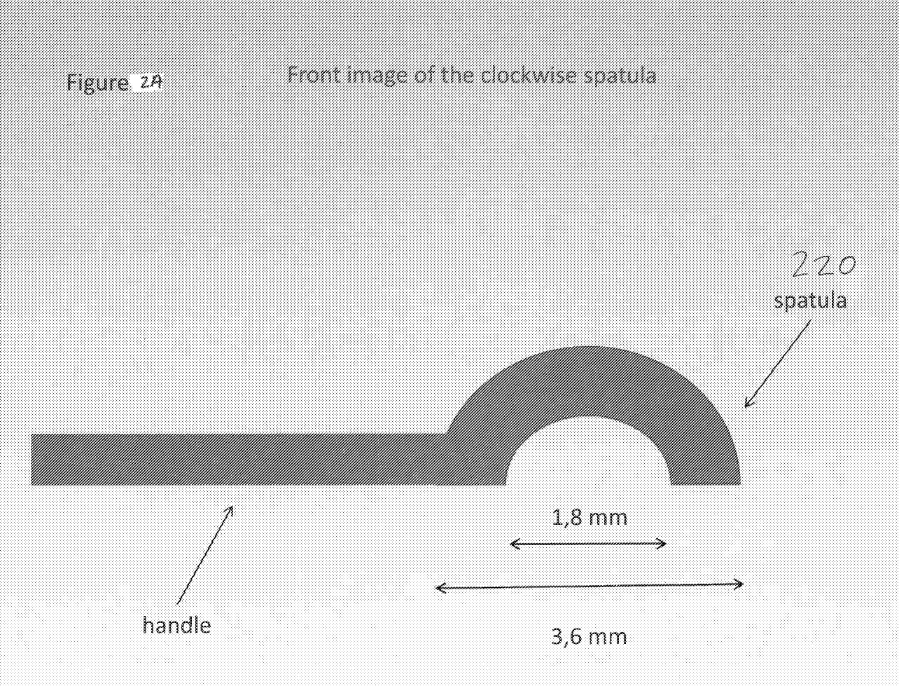

Once the ring 102 is complete, dye is injected through the ports 104, 106 to evenly distribute the dye in the ring volume. As shown in FIG. 3, with a syringe 344 and a “non-traumatic” needle 342, dye 340 is injected through the side ports 104 to evenly distribute the dye 340 in the ring 102 volume. In one example embodiment of the claimed invention, 0.5 mL was injected into each of the tunnels. The dye can be biochromaderm, biochromaderm ophtalmo, or other pigments and dyes approved for corneal use. Once the dye is injected and evenly distributed in the ring 102 volume, the surface of the cornea 108 is rinsed with balanced salt solution (BSS), and the lid speculum is removed.

The resulting cornea includes an intrastromal opaque ring centered on the visual axis of the cornea with a pinhole in the center of the ring. Near and intermediate vision is improved due to an increased depth of focus. Spectral domain OCT examinations demonstrate complete opacity of the dye. Histological analysis with hematoxylin and eosin stain confirms a continuous pigmented layer along the incision that does not diffuse in the adjacent stroma.

### Combined Procedure Embodiment

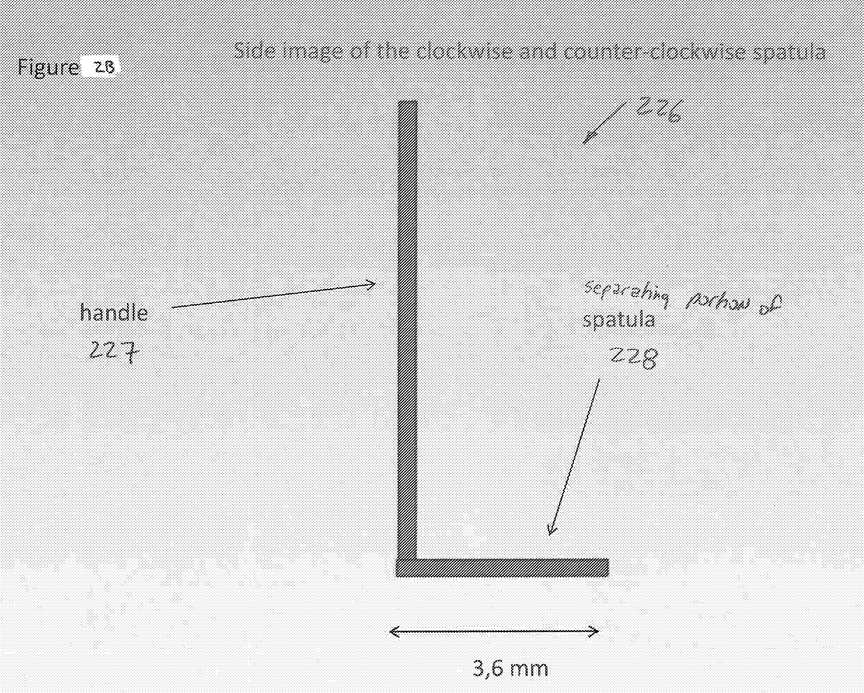

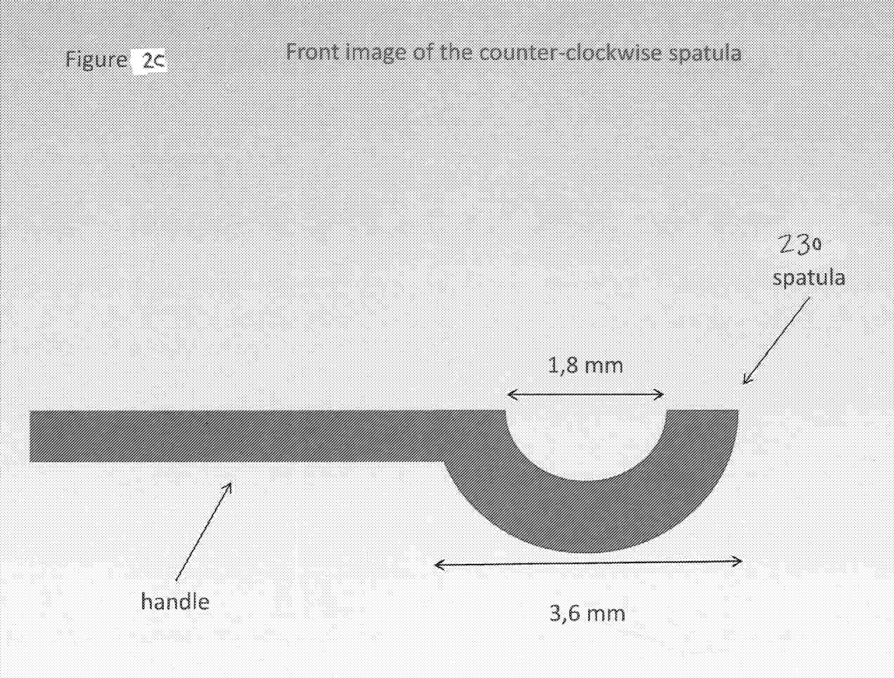

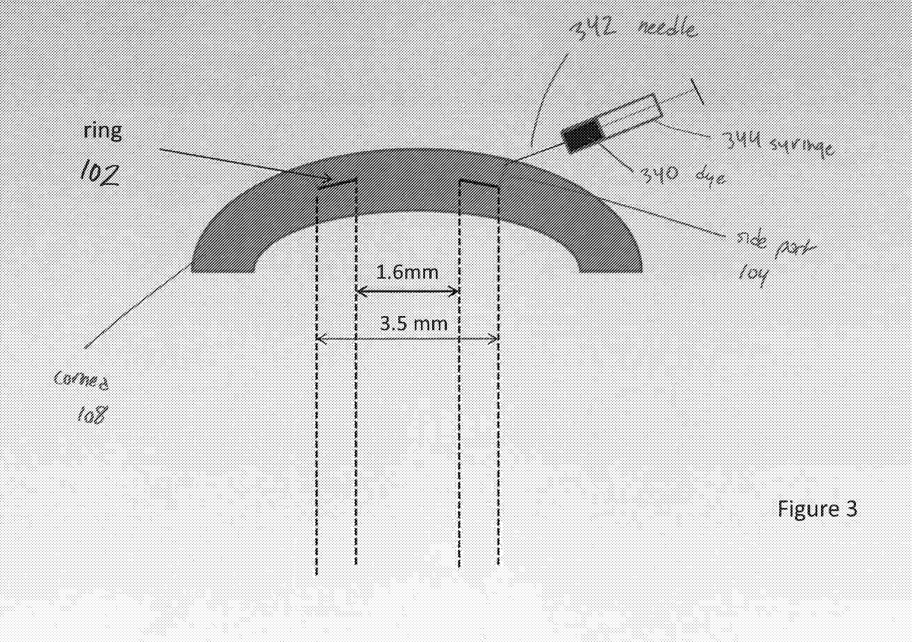

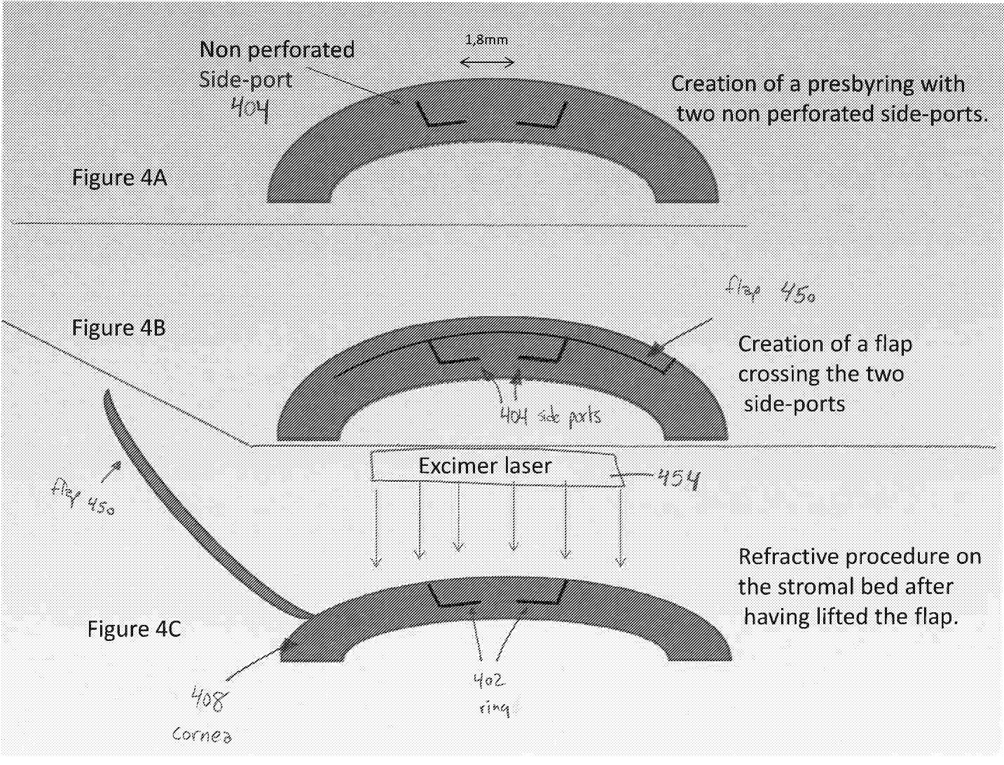

In another embodiment of the claimed invention shown in FIGS. 4A-4C, the PresbyRing method described above with regard to FIGS. 1-3 can be combined with a Lasik procedure to provide additional vision correction, such as treatment of a refractive error, including myopia, hyperopia, or astigmatism. For example, when the patient is positioned on the laser bed, a ring and one or two non-perforated side ports 404 (or radial ports) are created with the laser as shown in FIG. 4A.

In one embodiment of the claimed invention, the ring has a 1.6 mm internal diameter and a 3.8 mm external diameter. As above, the ring's depth can be between 5 microns and 500 microns deep, for example between 200 microns and 400 microns. The ring's dimensions can be modified depending upon the particular patient and presbyopia severity. As shown in FIG. 4B, a flap 450 is created with a femtosecond laser. The flap 450 intersects the two non-perforated ports 404. Once the flap is complete, the patient can be removed from the cone and placed under an excimer laser 454. As shown in FIG. 4C, the flap 450 is lifted and the refractive error is treated with the excimer laser 454. For example, the refractive error can be treated by reshaping the stromal bed of the cornea 408. Similarly, a PRK (photorefractive keratectomy) procedure can also be performed with an excimer laser by ablating a small amount of tissue from the corneal stroma at the front of the eye, just under the corneal epithelium.

In any case, after the refractive error is treated, two PresbyRing spatula devices in accordance with one example of the claimed invention are inserted through the two ports 404, which are now perforated since the flap 450 is lifted. As described above, FIGS. 2A-2C illustrate clockwise and counterclockwise spatulas in accordance with the claimed invention. By utilizing the appropriate spatula to perform each directional lift, the flap 450 is moved with less trauma. As before with regard to FIGS. 1D-1K, the two ring surfaces 402 of the cornea 408 created with the femtosecond laser are separated with spatulas in order to create a ring-shaped volume.

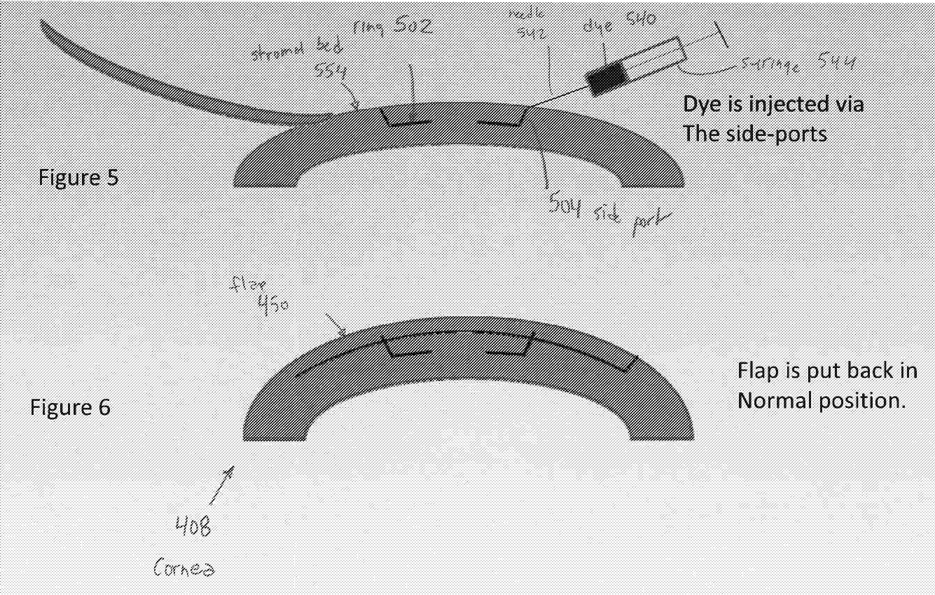

As shown in FIG. 5, once the ring 502 is complete, dye 540 is injected through the ports 504 to evenly distribute the dye in the ring volume. As shown in FIG. 5, with a syringe 544 and a “non-traumatic” needle 542, dye 540 is injected through the side ports 504 to evenly distribute the dye 540 in the ring 502 volume. In one example embodiment of the claimed invention, 0.5 mL was injected into each of the tunnels. The dye can be biochromaderm, biochromaderm ophtalmo, or other pigments and dyes approved for corneal use. Once the dye is injected and evenly distributed in the ring 502 volume, the stromal bed 554 is rinsed with balanced salt solution (BSS) and dried. As shown in FIG. 6, the flap 450 is folded back, the cornea 408 is rinsed with balanced salt solution (BSS), and the lid speculum is removed.

The resulting cornea includes an intrastromal opaque ring centered on the visual axis of the cornea with a pinhole in the center of the ring. Near and intermediate vision is improved due to an increased depth of focus as well as a reshaping of the cornea using Lasik or PRK processes. Spectral domain OCT examinations demonstrate complete opacity of the dye. Histological analysis with hematoxylin and eosin stain confirms a continuous pigmented layer along the incision that does not diffuse in the adjacent stroma.

### FemtoRing Embodiment

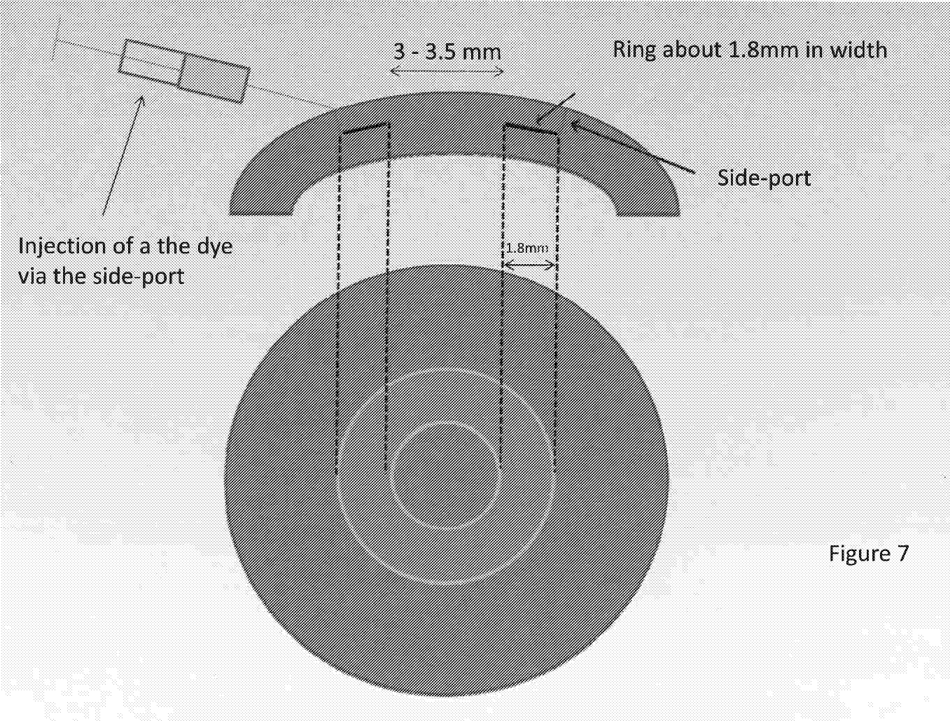

In another embodiment of the claimed invention, a similar technique can be used to create an intrastromal ring with the femtosecond laser. Different colored dyes can then be injected into the virtual space created with the laser in the cornea to change the eye color. For example, one method in accordance with the claimed invention includes positioning a patient on a laser bed. Topical anesthesia is given to the patient. The bed is moved under the femtosecond laser. A lid speculum is inserted, and the eye's cornea is brought in contact with the femtosecond laser's cone. As shown in FIG. 7, a ring and one or two side ports (or radial ports) are created with the laser. In one example embodiment shown in FIG. 7, the ring has a width of approximately 1.8 mm and is located in the mid-peripheral cornea. In other example embodiments, the ring has a width of approximately 1 mm. The ring's depth can be between 5 microns and 500 microns deep, for example between 200 microns and 300 microns. The ring's dimensions can be modified depending upon the particular patient and the efficiency and effectiveness in which the eye color changes, while allowing for sufficient examination of the eye.

After the ring and ports are created with the laser, the patient is removed from the cone and the eye is examined under a microscope. Two PresbyRing spatula devices in accordance with one example of the claimed invention are inserted through the two side ports. The PresbyRing spatula device can be a spatula as shown in FIGS. 2A-2C. As described above, FIGS. 2A-2C illustrate clockwise and counterclockwise spatulas in accordance with the claimed invention. By utilizing the appropriate spatula to perform each directional lift, the flap is moved with less trauma. The two surfaces created with the laser are separated using the PresbyRing spatula devices to create a ring-shaped volume.

As shown in FIG. 7, with a syringe and a “non-traumatic” needle, a colored dye is injected through the side ports in order to evenly distribute the dye in the ring volume. The dye is transparent enough to allow anterior and posterior segment examination. The dye can be biochromaderm, biochromaderm ophtalmo, or other dyes approved for corneal use. Different colored dyes or combinations of colored dyes can be used in order to change the eye color of the patient. The cornea is then rinsed with balanced salt solution (BSS), and the lid speculum is removed. With the dye inserted in the intrastromal ring of the cornea, the perceived color of the eye (that is, the iris) is changed.

### Removing the Dye

If the PresbyRing cannot be tolerated for any reason, it is possible to remove it using an excimer laser as shown in FIGS. 8A-8C. To remove the pigmented intrastromal ring, a corneal flap is created, laser ablation of the pigmented ring is performed, and the corneal flap is returned to its original position.

For example, a patient can be positioned on the laser bed, and a flap 1050 is created with a femtosecond laser as shown in FIG. 8A. The flap 1050 is created approximately 15 μm above the pigmented intrastromal ring (PresbyRing). For example, if the pigmented intrastromal ring 1004 is created at a depth of 150 μm, the flap 1050 is created at a depth of 135 μm. Once the flap 1050 is complete, the patient can be removed from the cone and placed under an excimer laser 1052. As shown in FIG. 8B, the flap 1050 is lifted and the pigmented intrastromal ring 1004 is treated with the excimer laser 1052. For example, the pigmented intrastromal ring 1004 can be removed by a 30 μm PTK laser ablation. After the ablation is performed, the flap 1050 is returned to its original position as shown in FIG. 8C.

### Example Embodiments of the Claimed Invention:

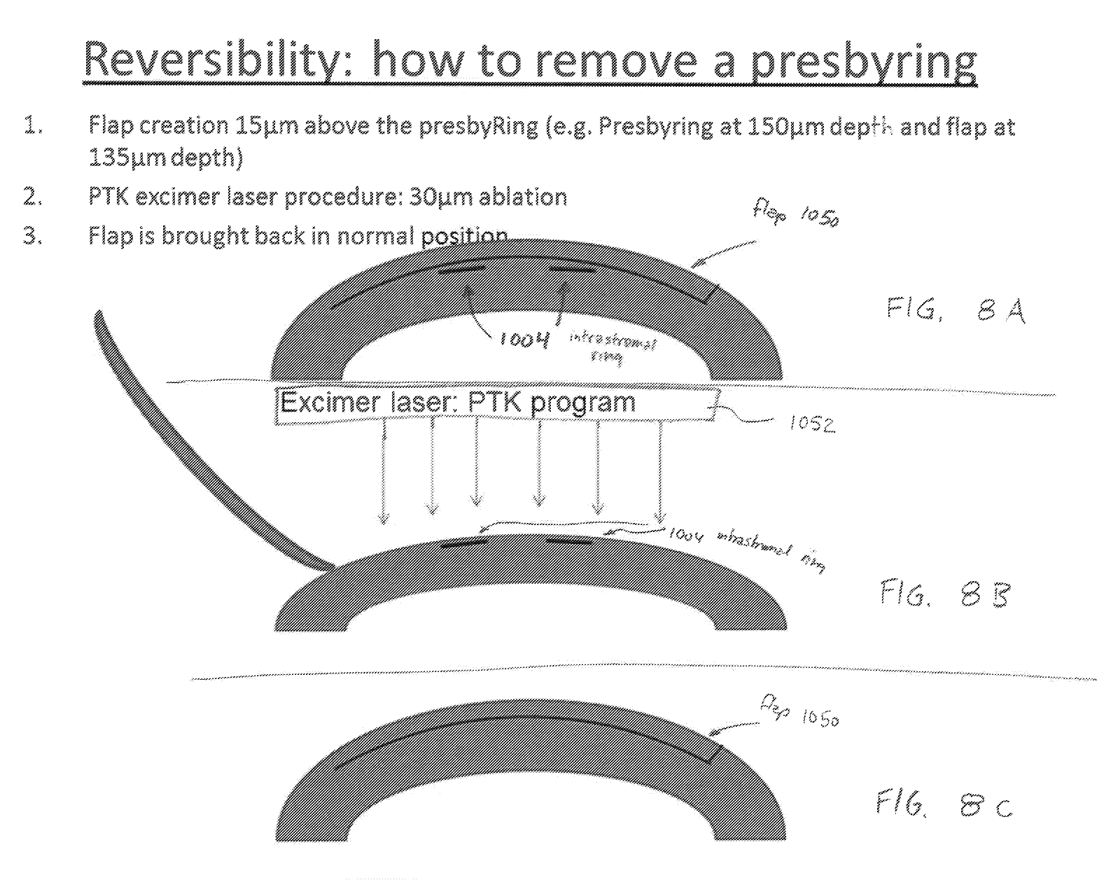

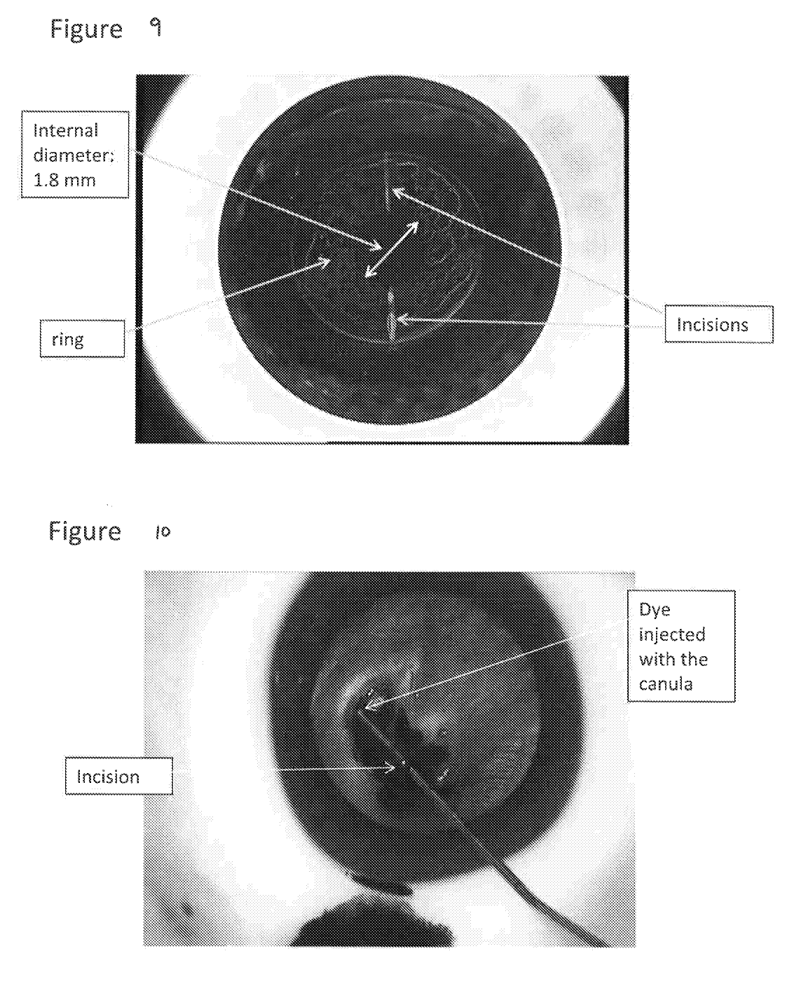

Five eyes of pigs, enucleated eight hours before the experiment, were used (Strasbourg slaughter house). A femtosecond laser (Visumax®, Jena, Carl Zeiss®) was used to create the surgical incisions. The eyes were treated with the Intra Corneal Ring program (ICR®) of a Visumax® laser to create tunnels for intracorneal rings (used for keratocones and the treatment of moderate myopia). The parameters of the tunnel diameters were changed as follows: internal diameter: 1.8 mm; external diameter: 5.1 mm (See FIG. 9). Three eyes (Eyes 1, 3, and 4 in Table 1) were programmed at a depth of 250 μm, one eye (Eye 2) was programmed at a depth of 150 μm and another eye (Eye 5) was programmed at a depth of 350 μm. One of the three eyes treated at 250 μm (Eye 1) served as a control and was not injected with the dye. The dye used in the other eyes was Biochromaderm®, which has meets CEIIb European medical standards. In the examples, one of the two half rings of the eye treated at 350 μm (Eye 5) was rinsed with a 3 cm³ syringe containing BSS, cannula for hydrodissection, two hours after injecting the dye, while the other pigmented half ring served as a control. Table 1 shows the procedure carried out in each of the eyes.

TABLE 1
Experimental Protocol Comparison

| | Surgical | Cutting | Dye | |
|---|---|---|---|---|
| Eye | Technique | Depth (μm) | Injection | Rinse |
| 1 | ICR | 250 | No | No |
| 2 | ICR | 150 | Yes | No |
| 3 | ICR | 250 | Yes | No |
| 4 | ICR | 250 | Yes | No |
| 5 | ICR | 350 | Yes | Yes |

After dissection of the tunnels, the black dye (Biochromaderm, Marseille, BioticPhocea®) was injected in the two tunnels thus created and was spread homogenously (FIG. 10). To do this, a 3 mL syringe and a cannula for the visco-elastic substance was used. With regard to the quantity of dye, 0.5 mL was injected into each tunnel. FIG. 11 shows the result of the procedure of the claimed invention where a ring is created using the femtosecond Visumax® laser, with the IntraCorneal Ring Program (ICR®) followed by the injection of the dye BioticPhocea® completely injected in the two tunnels.

All the eyes were examined using a slit lamp (Slit lamp Righton® RS-1000), magnifying×16, equipped with a Nikon® BM-6 camera and spectral domain optical coherence tomography to demonstrate complete opacity of the dye (OCT—HRA Spectralis, anterior segment lens VAO-00241 Rev. 3, mode IR+OCT section high resolution, angle 30°, rate 4.7/seconds, Heidelberg Engineering GmbH, Heidelberg, Germany). The eyes then underwent an anatomopathological analysis. Formalin fixation was done during 24 hours before paraffin embedding. Each paraffin block was cut with a micro-keratome so as to make cuts on the glass blades. The blades were colored with hematoxylin and eosin (HES) dye before analysis under an optical microscope.

The corneal examination by spectral domain OCT of the anterior segment enabled controlling of the depth and regularity of the stromal incision serving as a bed for the injection of the dye, as well as loss of corneal reflectivity related to the “mask effect” of the pigment. For the control eye, the interface of the incision was visualized at a depth of 221 μm. FIG. 12A shows Eye 1 treated with the IntraCorneal Ring Program at 250 μm without injection of dye. FIG. 12B shows diffused light and retro illumination, and FIG. 12C shows the OCT-SD spectrolysis results.

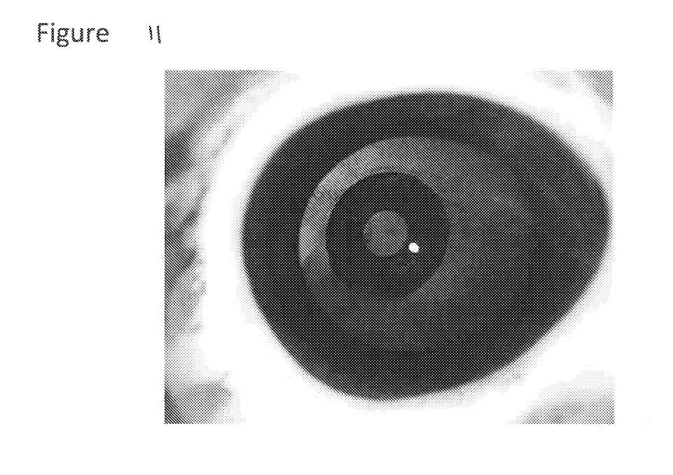

Histological analysis with hematoxylin and eosin stain highlights a continuous pigmented layer located along the incision, which does not diffuse in the adjacent stroma (FIGS. 13A-13B). A linear hyper-reflective line located along the incision interface and associated with a posterior shadow was revealed, indicating the totally opaque nature of the dye (FIG. 13C). No variation in corneal reflectivity was noted between the two half-incisions. For the eyes that were treated with the ICR® program at 150 μm and 250 μm (Eyes 1-4 in Table 1), the depth of the incision measured by spectral domain OCT was 135 μm for the eye programmed at 150 μm and 270 μm for the eyes programmed at 250 μm (FIG. 13D). The internal diameter was measured at 1750 μm.

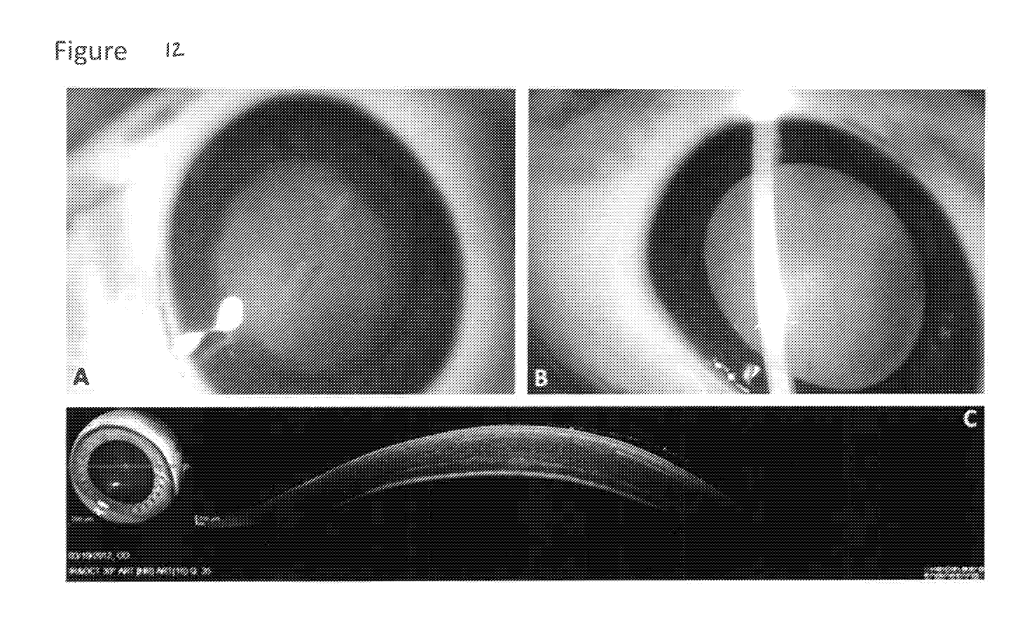

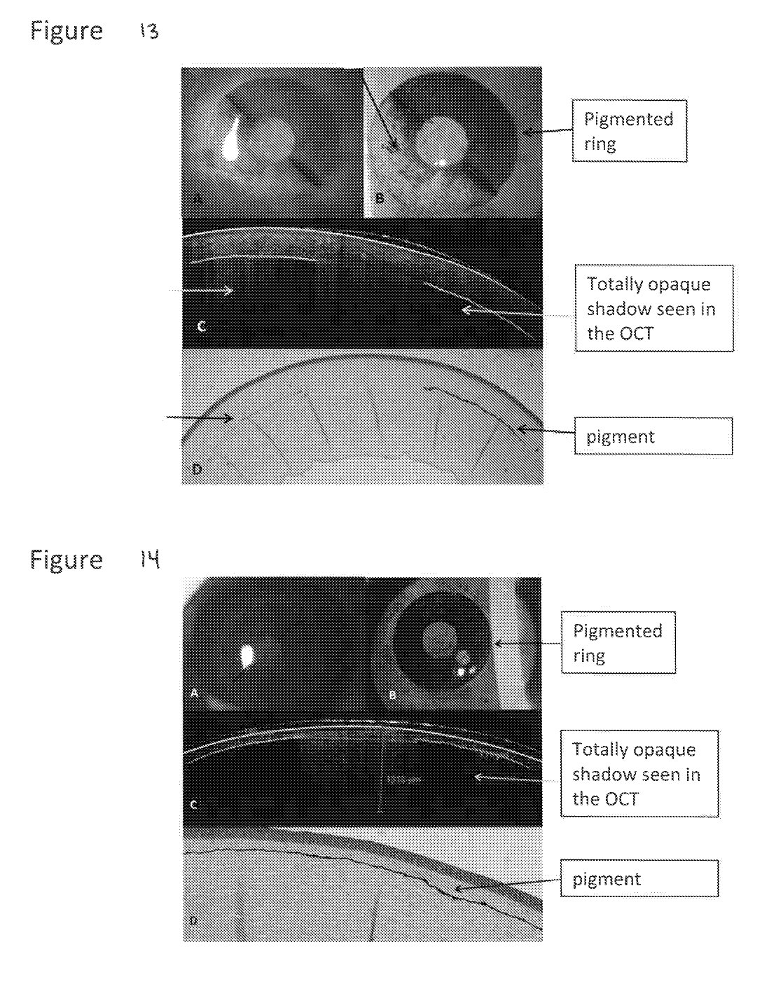

The stromal incision was measured at 420 μm for the eye that was treated with the ICR® at 350 μm (Eye 5 in Table 1). After the dye in the half-ring was rinsed out (about two hours after injection of the dye), examination under the slit lamp revealed that the intracorneal pigment in the half-ring had almost disappeared. A significant increase of the corneal reflectivity had been noted during the spectral domain OCT, in comparison with the half-ring that had not been rinsed out (FIGS. 14A-14D). FIG. 14B especially shows the result of the procedure of the claimed invention where a ring is created using the femtosecond Visumax® laser, with the IntraCorneal Ring Program (ICR®) followed by the injection of the dye BioticPhocea®. The half-ring of eye (Eye 5) was rinsed out 2 hours after injection of the dye. The diffused light, retro illumination, and the OCT-SD Spectralis are shown. The histological incision after hematoxyline eosin stain (HES) is magnified×25.

The anatomopathological analysis after HES staining reveals a clear decrease in the quantity of pigment of the rinsed half-ring compared to the non-rinsed half-ring. No spreading of the pigment was noted in the adjacent corneal stroma. The slit lamp also shows a distinct decrease in the quantity of dye in the rinsed half-ring.

The examples shown above create an intrastromal ring centered on the visual axis using a technique of keratopigmentation directed by a femtosecond laser. The results in FIG. 14C reveal a hyper-reflective blade in the spectral domain OCT located along the incision (corresponding to the dye), associated with a posterior shadow demonstrating the totally opaque nature of the dye, along with the absence of diffusion of the dye in the non-dissected stroma under the histological study. A discreet pre-descemetic hyper-reflectivity was observed in the control eye, in relation to the post-mortem time of the spectra domain OCT examination.

The rinsing test of the dye indicates a distinct decrease in the quantity of dye in the rinsed corneal stroma, compared to the non-rinsed corneal stroma, two hours after injection of the dye. This decrease was demonstrated by the spectral domain OCT and the anatomopathological examination. The anatomopathological and histological analysis with hematoxylin and eosin stain highlighted a continuous homogeneous distribution of the dye as the pigmented layer located along the incision, which did not diffuse into the adjacent stroma (FIG. 14D).

In comparison with previous techniques, the systems, devices, and methods of the claimed invention present a number of advantages, including the central functional pupil area is not dissected, there are no intracorneal foreign bodies, and the cost is much lower.

Having thus described the basic concept of the invention, it will be rather apparent to those skilled in the art that the foregoing detailed disclosure is intended to be presented by way of example only, and is not limiting. In addition to the embodiments and implementations described above, the invention also relates to the individual components and methods, as well as various combinations and sub-combinations within them. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as can be specified in the claims. Accordingly, the invention is limited only by the following claims and equivalents thereto.

1. A method of vision correction of an eye having a corneal epithelium, a Bowman's layer, and a corneal stroma, the method comprising:
forming an incision in the corneal stroma;
inserting a PresbyRing spatula into the incision in the corneal stroma to create at least one tunnel;
rotating the PresbyRing spatula in the corneal stroma to expand the at least one tunnel to create a ring-shaped volume;
inserting a dye in the ring-shaped volume to form a pigmented intrastromal ring.

2. The method of claim 1, wherein the incision is a side port incision.

3. The method of claim 1, wherein the incision is a radial port incision.

4. The method of claim 1, wherein the incision in the corneal stroma is created using a femtosecond laser.

5. The method of claim 1, wherein the ring-shaped volume in the corneal stroma is created using a femtosecond laser.

6. The method of claim 1, wherein inserting a dye in the ring-shaped volume includes injecting the dye.

7. The method of claim 1, wherein inserting a dye includes evenly distributing the dye in the ring-shaped volume.

8. The method of clam 1 further comprising:
rinsing the cornea with a balanced salt solution.

9. The method of claim 1 further comprising:
creating a corneal flap; and
folding back the corneal flap to provide access to the corneal stroma.

10. The method of claim 9 further comprising:
reshaping the corneal stroma with at least one of a femtosecond laser and an excimer laser.

11. The method of claim 1 further comprising:
performing a photorefractive keratectomy procedure.

12. The method of claim 1 further comprising:
removing the pigmented intrastromal ring.

13. The method of claim 12, wherein removing the pigmented intrastromal ring includes:
creating a corneal flap;
folding back the corneal flap to provide access to the corneal stroma;
ablating the pigmented intrastromal ring; and
returning the corneal flap to its original position.

14. The method of claim 13, wherein ablating the pigmented intrastromal ring includes ablating the dye that forms the pigmented intrastromal ring with an excimer laser.

15. A method of changing eye color of an eye having an iris, a corneal epithelium, a Bowman's layer, and a corneal stroma, the method comprising:
forming an incision in the corneal stroma;
inserting a PresbyRing spatula into the incisions in the corneal stromal ring to create a tunnel;
rotating the PresbyRing spatula in the corneal stromal ring to expand the tunnel to create a ring; and
inserting a dye in the ring to form a pigmented intrastromal ring.

16. The method of claim 15 further comprising:
distributing the dye in the ring.

17. The method of claim 15 further comprising:
rinsing the corneal stroma with a balanced salt solution (BSS).

18. The method of claim 15, wherein inserting a dye in the ring to form a pigmented intrastromal ring changes a perceived color of the iris.
